(19) United States
(12) Patent Application Publication
(10) Pub. No.: US 2013/0213443 A1
(43) Pub. Date: Aug. 22, 2013

(54) Method and apparatus for cleaning infant containers and accessories

(71) Applicants: Jorge Garcia, Edinburg, TX (US); Fernando Ramirez, San Juan, TX (US)

(72) Inventors: Jorge Garcia, Edinburg, TX (US); Fernando Ramirez, San Juan, TX (US)

(21) Appl. No.: 13/385,404

(22) Filed: Feb. 17, 2012

(51) Int. Cl. B08B 9/093; B08B 3/02

(52) U.S. Cl. 134/99.2

(57) ABSTRACT

An infant container cleaning apparatus is disclosed, comprising a stabilizer for holding one or more infant containers. The infant container cleaning apparatus also comprises a water application assembly for applying water, one or more cleaning agents, or a combination thereof to the one or more infant containers. Still further, the cleaning apparatus comprises at least one motion controller for varying the position, movement, speed, or a combination thereof of the stabilizer during the applying of the water, the one or more cleaning agents, or a combination thereof to the respective one or more infant containers by the water application assembly.

### BACKGROUND

Parents are constantly challenged to maintain clean and sterile baby bottles, pacifiers, nipples, teething rings and other items for use by their infants. Failure to properly clean or sterilize these items results in germ buildup that can cause infection or sickness. In the case of baby bottles, the cleaning task is further complicated by the presence of grooves, creases, differing bottle sizes and shapes and other factors; all of which make it difficult to reach and remove residue. Many people manually clean bottles using a cloth and soapy water, specialized scrub brushes for reaching the innermost parts of the bottle or even boiling. Alternatively, devices such as dishwashers or bottle sterilizers may be used. While sterilizers are designed to apply high temperature steam to the bottles as a means of purification, they do not typically perform any cleaning function. Conversely, dishwashers are configured to clean various items, but are not particularly suited to accommodate multiple bottles at a time or to provide sterilization. Also, many dishwashers fail to remove residue affixed to the bottle after completion of a wash cycle; resulting in manual cleaning being required. Unfortunately, there is currently no convenient, integrated system for effectively washing and sterilizing bottles and other infant items.

### BRIEF DESCRIPTION OF THE DRAWINGS

The embodiments of the invention are illustrated by way of example, and not by way of limitation, in the figures of the accompanying drawings:

FIGS. 1A-1C are diagrams of a system capable of enabling the cleaning of infant containers and other infant items, according to various embodiments;

FIG. 2 is a diagram of the components of a cleaning system, according to one embodiment;

FIG. 3 is a flowchart of a process for enabling the cleaning of infant containers and other infant items, according to one embodiment;

FIGS. 4A and 4B are diagrams of a mechanized container stabilizer and water application assembly of the system of FIGS. 1A-1C, according to various embodiments;

FIG. 5 is a diagram of an accessory cage for maintaining infant items to be cleaned and sterilized, according to one embodiment;

FIG. 6 is a diagram of a faucet configuration tool for directing water to the cleaning system of FIGS. 1A-1C, according to one embodiment;

FIG. 7 is a diagram of a digital control panel for enabling user control of the cleaning system of FIG. 1A-1C, according to one embodiment; and

FIG. 8 is a diagram of a chip set that can be used to implement an embodiment of the invention.

### DESCRIPTION OF SOME EMBODIMENTS

Examples of a method, apparatus, and computer program for cleaning infant containers and other infant items are disclosed. In the following description, for the purposes of explanation, numerous specific details are set forth in order to provide a thorough understanding of the embodiments of the invention. It is apparent, however, to one skilled in the art that the embodiments of the invention may be practiced without these specific details or with an equivalent arrangement. In other instances, well-known structures and devices are shown in block diagram form in order to avoid unnecessarily obscuring the embodiments of the invention.

As used herein, a “container” refers to any device for carrying or storing food, liquid or other items to be consumed by an infant, toddler or other child. Containers may include baby bottles, feeding tubes, jugs and cups of various dimensions, functions and material composition (e.g., Sippy cups). Also, as used herein, “cleaning” and/or “sterilizing” refers to any means of washing, disinfecting, sanitizing and otherwise removing bacteria, debris or hazardous particles from an item for hygienic, therapeutic or health purposes. For the purpose of illustration herein, cleaning and sterilizing are taken to be synonymous. Although various embodiments are described with respect to containers in the form of baby bottles, it is contemplated that the approach described herein may be used with other infant items including pacifiers, teething rings, toys, bottle tops and caps, cups, safety devices, utensils, nipples, breast pump equipment and the like.

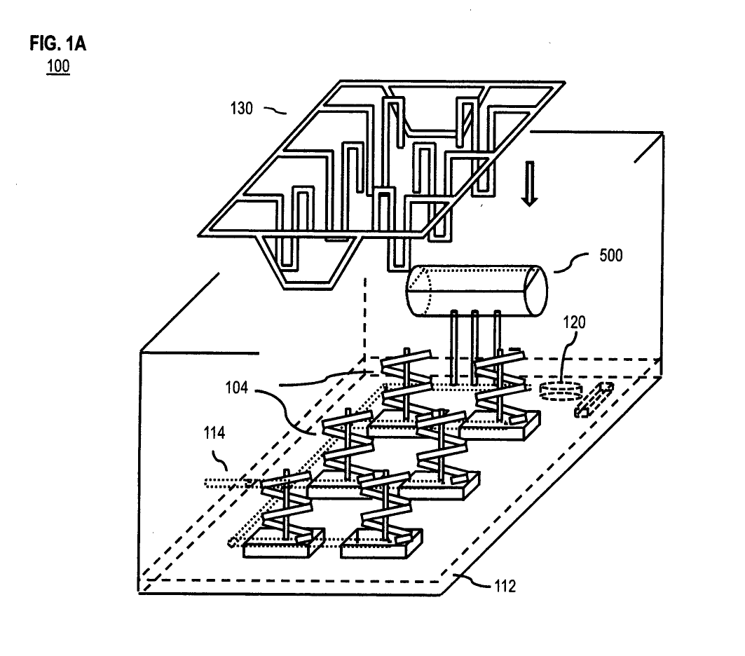

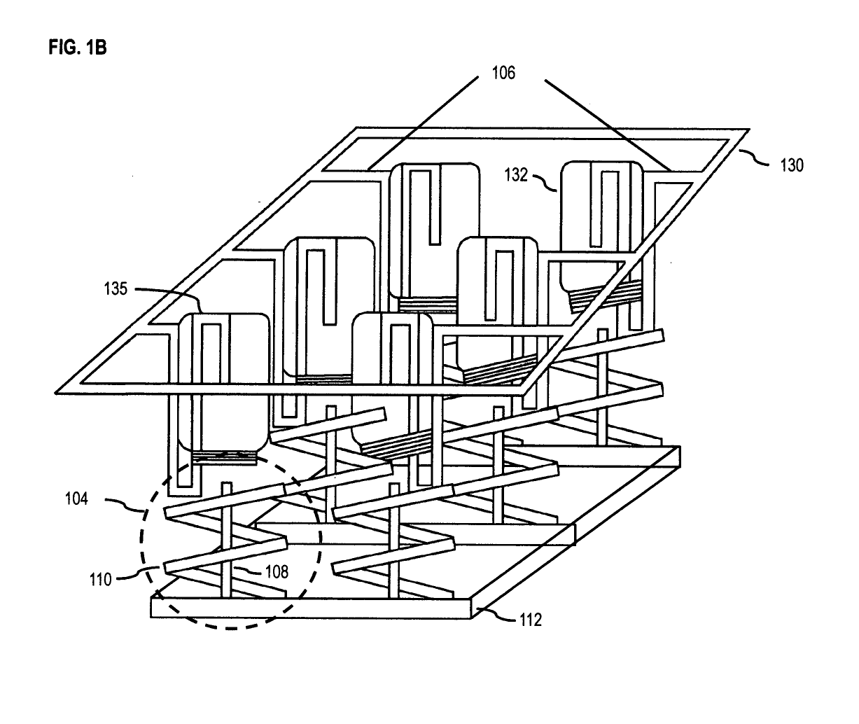

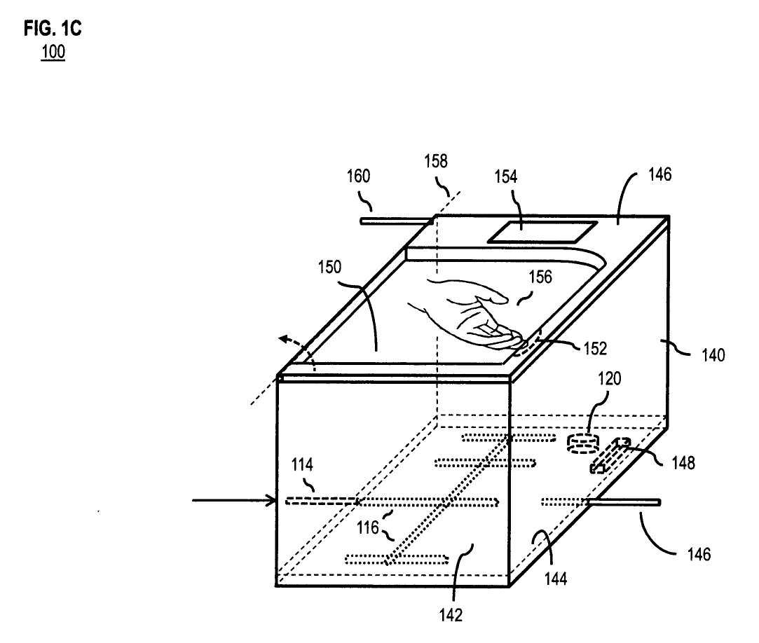

FIGS. 1A-1C are diagrams of a system capable of enabling the cleaning of infant containers and other infant items, according to various embodiments. By way of example, the system 100 is configured to clean multiple containers and infant accessories according to a multi-stage cleaning process. Typically, most parents, caretakers, nurses and other individuals that care for infants, toddlers and other children clean containers and other items manually. The manual cleaning process may involve the use of a wash cloth, sponge, brush or specialized tool designed to reach into and around the container (e.g., a baby bottle). Alternatively, the person may resort to vigorously shaking the bottle as it is filled with soap and water, so as to remove or loosen residue. Still yet, in other instances, the items may be boiled as a means of sterilization or cleansed with special detergents. Unfortunately brushes, wash cloths and other tools can harbor bacteria, which in turn get deposited onto the items being cleaned. Also, vigorous shaking of the bottle is tedious and does not ensure the removal of all residue deposits and other buildup accumulated to the surface walls. Moreover, boiling can cause deformity of the item, the potential release of toxins (e.g., Bisphenol A (BPA)) or other types of damage.

Alternatively, devices such as dishwashers or specialized bottle sterilizing equipment can be used to clean containers such as baby bottles. Generally, sterilizers are designed to apply high temperature steam to the bottles as a means of disinfection. They do not, however, perform any cleaning function, which limits the usefulness of the device as an overall cleaning solution. In contrast, dishwashers are configured to clean items but are not particularly suited to accommodate bottles or perform sterilization. Some dishwashers even fail to remove residue and other buildup affixed to the bottle even after completion of a wash cycle, especially residue that is trapped near the inner bottom edges of the bottle or in the grooves at the top of the bottle where the cap is secured. The result is additional, often manual cleaning being required to render the bottle in a good state. Unfortunately, there is currently no convenient, integrated system for effectively washing and sterilizing bottles and other infant items. Furthermore, there is no automated device or mechanism that is configured to alleviate hard to reach residue build up without requiring manual cleaning.

To address this problem, a system 100 of FIG. 1A and FIG. 1C introduces the capability to automate the internal and external cleaning of bottles and other infant items through execution of a multi-stage cleaning approach. By way of example, the cleaning approach is executed automatically and programmatically, such that a user may customize the cleaning approach to their preferences. The system 100 is also configured to facilitate movement of one or more bottles as water and/or other liquid (e.g., detergent) is applied to the bottles concurrent with execution of the various stages of cleaning; the movement causing variable application of water to various inner and outer points along the surface of the bottle to enhance removal of hardened, viscous or wet food, formula, juice, etc.

In certain embodiments, the system 100 (e.g., cleaning system) includes a mechanized container stabilizer 130 for holding the bottles in position between and around one or more stems of a water application assembly 112. For the purpose of illustration, a sprayer 104 refers to a complimentary set of stems, including a central and spherical stem respectively, for concurrently cleaning the inside and outside of a bottle. Also, for the purpose of illustration, the water application assembly 112 includes a group of interconnected sprayers 104. As will be discussed more fully later on, the sprayers 104 are interconnected by way of a common channel or network of waterways (e.g., tubes, stems or the like) for distributing water, cleaning agents maintained in a reservoir or dispenser 120, or a combination thereof to the various bottles.

The mechanized container stabilizer, hereafter referred to as a stabilizer 130, enables loading of multiple bottles for concurrent cleansing by the system 100 as they are positioned adjacent to and between respective sprayers 104. For example, FIG. 1B is a diagram depicting the stabilizer 130 loaded with six bottles (e.g., bottles 132 and 135) of varying size, dimensions or combinations thereof; each bottle being aligned adjacent to a corresponding sprayer 104. By way of example, the bottles are placed into the stabilizer 130 in a downright position for exposing their respective openings to the sprayers. The bottles are loaded onto the stabilizer 130 via one or more placement arms 106, which make contact with the inner and outer surface of the bottle for holding it downright. The stabilizer 130 may be lowered such that a central stem 108 of a respective sprayer 104 extends into the opening of the bottle 135. As such, the stem 105 sprays water to an inner portion of the bottle as forced through one or more water holes extending along the stem 108.

Each sprayer 104 also comprises a spherically shaped stem (e.g., spherical stem 110) that, similar to the central stem 108, extends upward from the base of the water application assembly 112 towards a corresponding bottle 135. The spherical stem 110 envelops the bottle 135 as it is lowered, thus enabling the application of water, soap or any other cleaning agent to the outside of the bottle 135. The circumference of each spherical stem 110 is such that it encompasses the outer portion of the bottle 135 to accommodate bottles of varying sizes and shapes.

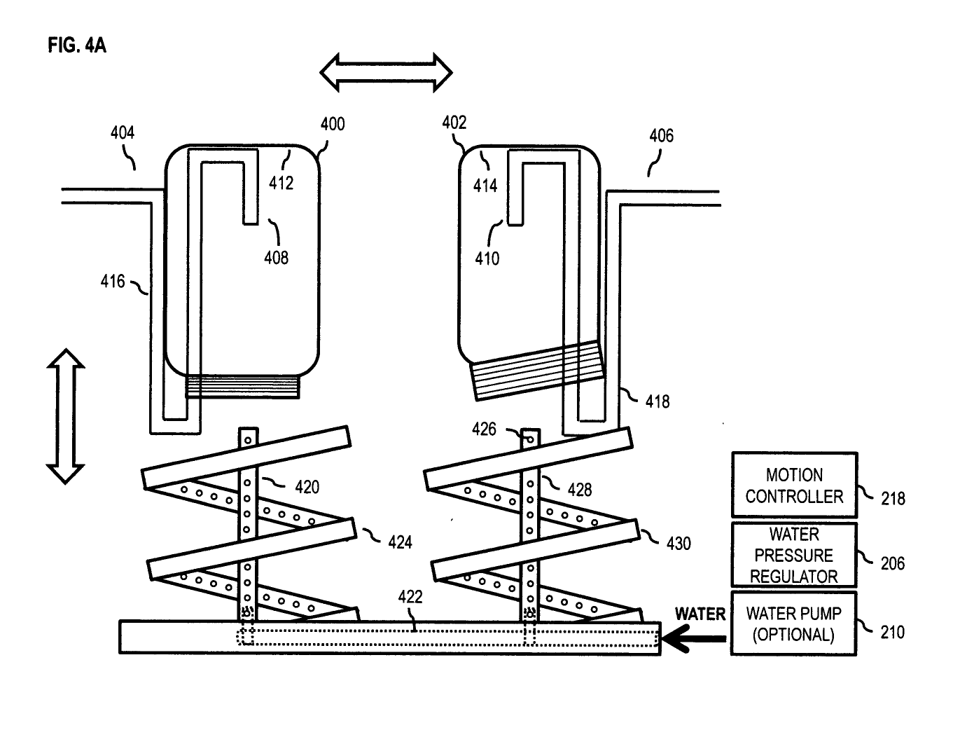

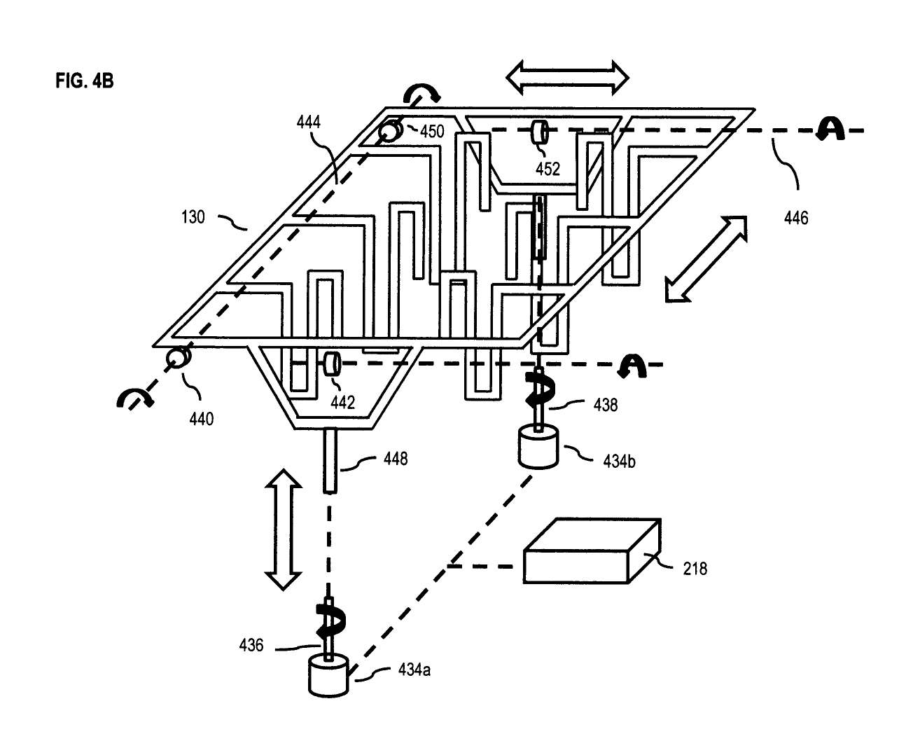

It is noted that water is forced onto the inner and outer portions of the bottle by way of several holes that extend along the central stem 108 and spherical stem 110 respectively. Also, water is brought into the system 100 from a water supply by a water inlet 114, shown by way of example in FIG. 1C. The water flows from the inlet 114 through various water ways 116 that extend throughout the water application assembly 112. The water inlet 114 connects to a water supply, such as provided by a faucet, water spout, hose, etc. Hence, the water application assembly interacts with the container stabilizer 130 to enable cleansing of bottles. As will be described more fully later on with respect to FIGS. 4A and 4B, the position, movement and/or speed of the stabilizer 130 may be varied during various stages of the cleaning process enacted by the system 100. This variation affects how the water application assembly 112 directs water to the bottle to enable cleaning.

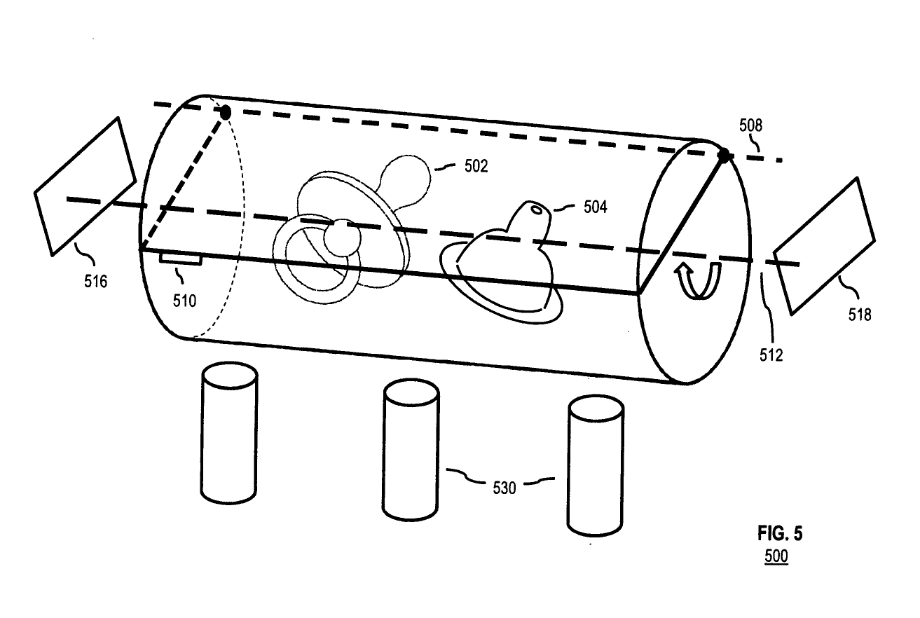

In one embodiment, the system 100 also includes an accessory cage 500 configured to house pacifiers 502, teething rings, nipples 504, bottle tops/caps and other infant items. FIG. 5 is a diagram of an accessory cage for maintaining infant items to be washed and sterilized. A user may load infant items to be cleaned by lifting a top portion of the cylindrically shaped accessory cage, as represented by opening seam line 506. The top portion of the cage 500 pivots about an axis 508 (e.g., a seam axis). Upon releasing of a seal latch 510 or a manually forced lift, the top portion of the cylinder is opened about the axis 508. It is noted that the seam axis 508 is parallel to a center axis 512 that extends through the center of the entire cage 500.

The accessory cage 500 may be implemented in a mesh or netted design for enabling the passage of water from one or more waterways 530 (e.g., stems or hoses) to the items 502 and 504 loaded therein. Water may be forced to the cage by way of a water pump, pressure regulator or other means.

In certain embodiments, the accessory cage 500 is supported by a first and second extension pin, each pin extending from a first and second wall of the system 100 housing through the center axis. By way of example, a portion of the first and second wall 516 and 518 that the pin would contact is shown. The accessory cage is also offset from the stabilizer and/or water application assembly, so as to not impede execution of the cage. Of note, the accessory cage can be configured to spin about the center axis extending through the extension pins, wherein the accessory cage is caused to spin due to water pressure, through automation means, or a combination thereof.

It is noted that the accessory cage 500 facilitates the cleaning of accessories concurrent with the cleaning of bottles. As such, the loading of bottles to the stabilizer 130 may be performed concurrent with the loading of any other infant items to the accessory cage 500 during a loading stage of the multi-stage cleaning cycle.

FIG. 1C is a diagram depicting a housing of the system 100, the housing comprising one or more walls, including various upright enclosures (e.g., walls 140 and 142), a flooring 144 for supporting the water application assembly 112 and a top enclosure 146 that functions as a door. The water application assembly 112 is affixed to the bottom (e.g., flooring 144) of the cleaning system 100, where it interconnects the sprayers of the water application assembly via a network of waterways 116 (e.g., hoses or tubes). As mentioned previously, the network of water ways 116 enable the flow and distribution of water to each sprayer—the water being distributed equally by way of various mechanical and/or electrical pressure regulation techniques. Water flows into the system 100 through the inlet 114 and excess or unwanted water is expelled through a drain 146.

In certain embodiments, the flooring houses or has affixed thereon various components and accessories of the system 100, including a soap reservoir 120, water reservoir 148, additional tubing 116, electronic circuitry, power supply for accommodating an insulated power cord 160, etc. By way of example, all or some of the components and accessories may be affixed to the flooring 144 alongside the water application assembly 112 or integrated within. Resultantly, these components are rendered visible to a user when observed from an overhead perspective. Alternatively, all or some of the components and accessories may be affixed to the flooring 144 or integrated into the water application assembly 112 in a concealed manner, where they are not viewable by a user from an overhead perspective.

In one embodiment, the upright enclosure includes four walls, including wall 142 and its complement as well as wall 140 and its complement. Each wall is affixed to the flooring 144, thus enclosing the water application assembly 112, stabilizer 130, accessory cage 500 and other internal components of the system from the bottom and sides. The housing further comprises a top enclosure 146, which by way of example, acts as a door for enabling the user to enclose the system. By way of example, closing of the door ensures a self-contained cleansing environment for ensuring optimal cleansing and sterilization. One or more vents may also be placed at various locations along the housing, such as to enable ventilation of the bottles with respect to a given cleaning stage.

The door 146 may feature a viewing pane 150, constructed of an opaque material that enables the user to observe the cleaning process as it is performed. When ready, the user may lift the top enclosure (opens the door 146) by exerting an upward force at a handle, slot 152 or concave opening, by activating a latch or button at a user control panel 154, or by other means. Lifting or releasing of the top enclosure 146, such as by hand 156, causes it to pivot about a pivot axis 158 for rendering the opening or closing of the top enclosure.

In certain embodiments, the user may observe the internal components of the system 100 when viewed through the viewing window 156, including operation of the various components during execution of the system. The door 146 may also feature a control panel interface 154 by which the user controls various functional and operational aspects of the system 100, i.e., the number or sequence of cleaning stages, etc. Under this scenario, as an overhead functioning door 146, the aesthetic, functional and operational characteristics of the top enclosure are suited for accommodating an overhead perspective. In other embodiments, however, different approaches may be employed, including placement of the viewing window and/or control panel along a wall 140. It is noted, for the exemplary configuration of FIG. 1C, that the door146 is the single point of opening or closing of the system 100. Also, while not expressly shown, a sensor may be configured to detect the active or inactive positioning of the top enclosure 146 (e.g., latch open or closed condition), such as to enable or disable operation of the system based on the determined position.

As shown by way of example in FIGS. 1A and 1C, the system 100 is implemented in a rectangular form that features a single entry point to the stabilizer 130, water application system 112, etc. by way of the top enclosure 146. In this approach, the user loads bottles (e.g., bottles 132 and 135) into the stabilizer 130 from overhead. The user also loads any infant items requiring cleaning into the accessory cage 500 from overhead. Once loaded, the user then closes the top enclosure 146 to trigger the execution of a predetermined cleaning process, or alternatively, enable the user to manipulate system execution via the control panel interface 154. When the top enclosure 146 is closed, the housing is self-contained to prevent or minimize the entry of air, water or heat unless required in connection with an executed cleaning stage.

In the example depicted herein, the rectangular shaped system 100 may be suitably placed atop a countertop, sink base or other surface area for enabling the user to readily load infant containers and accessories while within proximity of a water supply (e.g., faucet) and drainage source. Alternatively, the cleaning system 100 may be suitably positioned into a sink basin. It is noted that various other means of implementation of the upright enclosure (e.g., one or more walls), flooring and top enclosure (e.g., door 146) may be used. By way of example, the configuration of the housing, stabilizer, water application assembly or other internal components of the system 100 may be adapted to other shapes and form factors. Under this scenario, the stabilizer 130, water application assembly 112, housing, etc., may be cylindrically shaped.

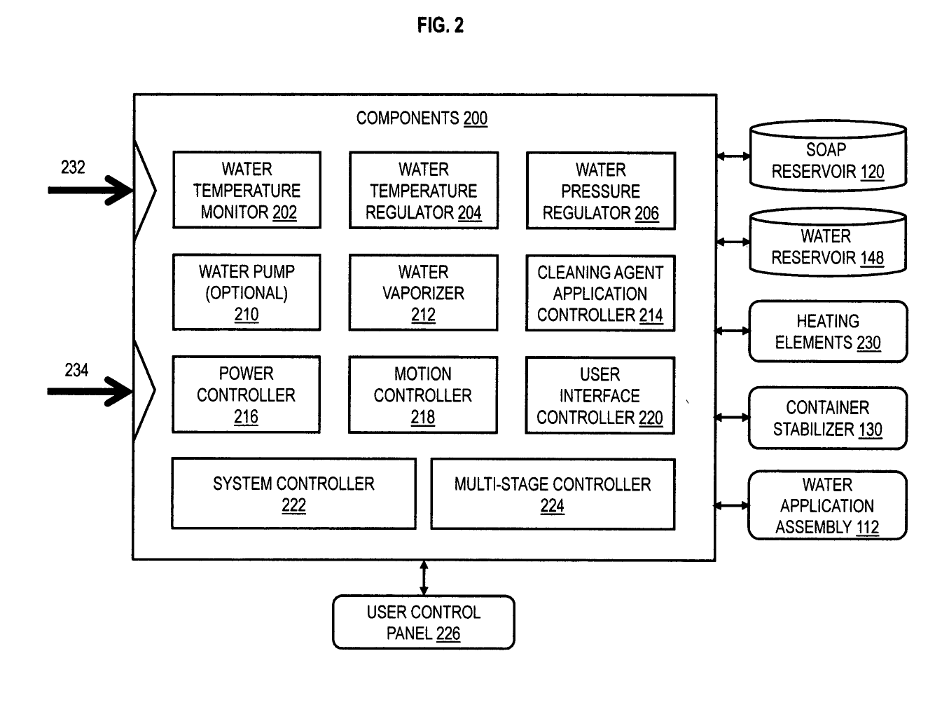

The cleaning system 100 includes various elements and corresponding components that interact to enable multi-stage cleaning of bottles, infant accessories, etc. FIG. 2 is a diagram of the components of the cleaning system, according to one embodiment. Various elements of the system 100, such as the stabilizer 130, water application mechanism 112, heating elements 230, water reservoir 148, soap reservoir 120, a user control panel 226 and the like may be implemented as one or more mechanical, electrical, electro-mechanical, static elements or a combination thereof. In certain embodiments, various executable components 200 may be implemented in hardware, firmware, software, or a combination thereof for facilitating interaction between these elements to ensure cleaning of infant containers. It is contemplated that the functions of these components may be combined in one or more components or performed by other components of equivalent functionality.

By way of example, the components 200 of the cleaning system 100 may include a water temperature monitor 202, a water temperature regulator 204, a water pressure regulator 206, a water pump (optional) 208, a water vaporizer 212, a cleaning agent application controller 214, a power controller 216, a motion controller 218, a user interface controller 220, a system controller 222 and a multi-stage controller 224. In addition, the various components 200 access operational data from a data store (not shown) for executing one or more programmatic instructions required of the system 100. The operational data may include user specified input data for affecting operation of the system, default operation data, etc.

For the purpose of illustration, any action, function, or operation carried out by a particular component with respect to an individual sprayer 104 of the system 100 may also apply to the water application assembly. Likewise, the subsequently described actions, functions, or operations of the components 200 may also be carried out in a similar manner for cleansing items maintained in the accessory cage 500 (as shown in FIG. 5).

In one embodiment, the water monitor 202 keeps track of the temperature of water as input to the system 100 via a water supply 232 and as it circulates throughout the various water ways of the system. The water monitor 202 is also configured to monitor water pressure rates as at various points throughout the system. By way of example, the water temperature monitor 202 may determine that the incoming water temperature, as provided by the water supply 232 (e.g., a kitchen faucet), is below a preferred cleaning temperature. Based on this measure, the monitor 202 may trigger operation of a water temperature regulator 204, which is configured to adjust the water temperature to a desired setting via activation of one or more heating elements 230.

In one embodiment, the one or more heating elements 230 are placed at various points within the cleaning system 100 for enabling water to be heated to a recommended/desired/programmed temperature. By way of example, heating elements 230 may be placed near a water reservoir 148 or along the various water ways of the water application assembly 112. The desired or default temperature setting may for heating the water may correspond to 130-140 degrees Fahrenheit—i.e., a recommended temperature for cleaning items. In certain embodiments, the heating elements 230 may be implemented in various forms for accommodating differing system 100 form factors and configurations. This may include, for example, electrically charged coils, ceramic components, heating plates and the like. Of note, the water monitor 202 oversees the heating process and then alerts the temperature regulator 204 when the desired temperature has been met. It is noted also that in certain instances, the cleaning system 100 may be caused to delay one or more cleaning stages depending on the determined temperature. Under this scenario, the cleaning stage is only allowed to proceed when the desired temperature is met.

In one embodiment, a water pressure regulator 206 may also operate in connection with the water monitor 202 for conditioning the water pressure brought into the system 100 from the water supply 232. By way of example, the water monitor 202 may determine a current water pressure or intake quantity/rate is not suitable for ensuring proper application of water to bottles and other items. Based on this reading, the water monitor 202 triggers operation of the water pressure regulator 206; which is configured to regulate the opening, closing and/or restricting of an intake valve of the system. Intake valves may be placed at various points throughout the system, including at a water inlet, at a base of the water application assembly 112, etc., to constrict or expand the flow of water as required. Of note, the water monitor 202 oversees the regulation process and then alerts the pressure regulator 206 when the desired pressure has been met. It is noted also that in certain instances, the cleaning system may delay one or more cleaning stages depending on the detected pressure, and only allow the cleaning to proceed when the desired pressure is met.

In one embodiment, a water pump 210 (optional) may be configured to propel water through the water application assembly 112 for impacting the rate at which water is forced onto bottles and other infant items. Of note, the water pump 210 may operate in conjunction with the water monitor 202 and pressure regulator 206 to direct variable pressure or controlled pressurized water to the bottle internally and externally. Depending on cleaning requirements, the water pump 210 may be programmed to vary the pump rate as a means of further removing residue and other unwanted deposits on bottles and other infant items. By way of example, the pump may be driven by an electric motor for enabling the following:
During the pump cycle, the pump 210 forces water into the stems (e.g., central and spherical stems) at a certain rate;
During the drain cycle (e.g., drying stage), the pump 210 directs the water into the drain hose.
   Of note, the motor-pump assembly may be configured for reversible operation, uni-directional operation (non-reversible), forced flow operation or the like to impact water application.

In another embodiment, a water vaporizer 212 causes production of vaporized water for enabling the sterilization of the one or more bottles. By way of example, the water vaporizer 212 produces a control signal for causing one or more of the various methods of vaporization of TABLE 1 below:

TABLE 1
Exemplary water vaporization approaches

Steam vaporization as produced from the boiling of water by one or more
heating elements 230. By way of example, the water vaporizer 212 causes
heating elements 230 to slowly heat up the water until the water changes
states from liquid to gas in the form of steam.
Impeller vaporization may be caused by the water vaporizer 212 as a result
of breaking water into microscopic droplets and propelling them into the
self contained housing. Under this approach, the items to be cleaned are
encompassed by vapor.
Ultrasonic vaporization may be caused by the water vaporizer 212 through
the use of ultrasonic frequency to create water mist. The speed of vibration
increases until the speed is fast enough to separate the water into small
droplets. These droplets are then propelled into the self contained housing
environment in a manner similar fog to impeller based vaporization.

Regardless of the approach employed, the water vaporizer 212 can be configured to direct water vapor through the central and spherical stems of each sprayer (e.g., or of the entire water application assembly) for enabling internal and external vapor build up. Likewise, the water vaporizer 212 may direct the vapor accordingly to any items maintained in the accessory cage 500. It is noted that the sterilization process may require a vapor release action be performed during the sterilization stage of the cleaning process, such as to prevent excess vapor buildup.

In one embodiment, a cleaning agent application controller 214 disperses soap or any other cleaning agent from the soap reservoir 120 in a controlled manner. Soap may be applied by the cleaning agent application controller 214 in accordance with a predetermined cleaning sequence, user specified commands, or a combination thereof. Also, in one embodiment, a power controller 216 regulates the application of electrical energy throughout the system 100 as supplied by a power supply 234.

In one embodiment, a motion controller 218 regulates the motion (e.g., direction and/or speed) of the container stabilizer 130 and thus the one or more infant containers (e.g., bottles). As such, the pattern of water applied to the inner or outer surface of the bottles via the various outlets of the spherical and central stems is varied. The variation enhances the ability of the water application assembly 112 to remove or loosen residue accumulated to the bottles during the various stages of the cleaning process. For example, a change in speed, motion and/or position of a bottle may correspond with a varied angle of attack for dislodging of viscous residue. More regarding operation of the motion controller relative to the stabilizer and/or water application assembly is described with respect to FIGS. 4A and 4B, according to various embodiments.

In FIG. 4A, bottles 400 and 402 of different types are shown to be loaded onto respective placement arms 404 and 406 of the stabilizer 130. In one embodiment, the placement arms 404 and 406 are configured in the shape of a hook for holding bottles in place within the stabilizer 130 and during the cleaning process. The bottles are maintained in place by resting the bottles 400 and 402 against various portions of the placement arms 404 and 406 with the bottled in a downright position. For example, a portion 408 and 410 of placement arms 404 and 406 respectively make contact with the inside of bottles 404 and 406. Under this scenario, portions 408 and 410 of the placement arms makes contact with at least the base 412 and 414 of respective bottles 400 and 402. In addition, outer portions 416 and 418 of the placement arms 404 and 406 respectively are positioned to make contact with an outer surface of the bottle. It is noted that the inner 408/410 and outer 416/418 portions of respective placement arms 404 and 406 may exert a tensional force against each other. Resultantly, this causes bottles 400 and 402 to be lodged in place within the stabilizer 130 to preventing shifting or movement. The stems may be of a length suitable for accommodating differing sizes, shapes and lengths of bottle. In addition, various other configurations of placement arms may be used to facilitate the holding, stabilizing, raising or lowering of the bottles 400 and 402.

As mentioned, each sprayer comprises a spherically shaped stem 424 and central stem 420 that extends upward from the base of the water application assembly. The base houses a network of water ways 422 for enabling water to be routed through the stems 420 and 424. Water is forced through the water ways 422 to the stems, such as by way of a pump 210 and/or water pressure regulator 206. The water is then forced through several outlets (e.g., 426) that extend along the stems to contact the bottle. While not shown, it is noted that the outlets may extend along the inner and outer surface of the stems 420 and 424 for enabling multi-directional spraying of the water and/or cleaning agent.

Once the bottles are loaded accordingly, the motion controller 218 is activated. This results in a lowering of the stabilizer and increased interaction between the bottles 400 and 402 and a respective sprayer. By way of example, as the placement arms 404 and 406 of the stabilizer 130 is lowered, corresponding central stems 420 and 428 enter the opening of bottles 400 and 402. Under this scenario, the stems 420 and 428 extend more deeply inside bottles 400 and 402. Likewise, as the bottles are lowered, the corresponding spherical stems 424 and 430 of respective sprayers increasingly encompasses the outer surface of bottles 400 and 402. It is noted that the placement arms 404 and 406 may be configured to fit within the circumference of the spherical stems 424 and 430; thus enabling substantial lowering of respective bottles 400 and 402 to accommodate differing bottle lengths.

In FIG. 4B, the stabilizer 130 is shown to interact with at least one motion controller for enabling variable direction or speed of the stabilizer. By way of example, the motion controller 218 may include a rotational motor/drive mechanism 434a and 434b for controlling one or more extension rods 436 and 438 that extend toward and/or affix to various points of contact along the stabilizer 130. The vertically placed extension rods 436 and 438 may connect to a female threaded shaft 448 located along a middle point (e.g., handle portion) at both sides of the stabilizer 130. The extension rods 436 and 438 are threaded complimentary to the female threaded shafts (e.g., 448), such that as respective motors 434a and 434b are driven by the motion controller 218, the extension rods 436 and 438 increase or decrease in a vertical length accordingly. This motion corresponds to that required for enabling downward movement of the loaded bottles towards a respective sprayer, or upward movement of the bottles such as upon completion of the cleaning process. This motion may also be used for facilitating movement of the bottles during a selecting cleaning stage or cycle of the system 100.

As another example, the motion controller 218 may also control one or more motor driven threaded rollers (e.g., 440 and 442) configured to make contact with various portions of the stabilizer 130. The points of contact of the stabilizer relative to the position of the rollers (e.g., 440 and 442) may be correspondingly threaded, such that rotational movement of the rollers enable horizontal and/or forward movement of the stabilizer 130 as the adjacently placed threads make contact. Under this scenario, a first 440 and second 450 threaded roller is positioned along an axis 444 for making contact with a portion of the length of the stabilizer. Driving of the first 440 and second 450 threaded roller corresponds to a back and forth motion of the stabilizer 130. A third 442 and fourth 452 threaded roller is positioned along an axis 446 for making contact with a portion of the width of the stabilizer, such that driving of the third 442 and fourth 452 rollers corresponds to lateral motion of the stabilizer 130.

It is noted that the motion controller 218 enables at least two types of motion for affecting various cleaning stages of the bottles, including: (1) loading and unloading motion, such as for regulating the up/down motion of bottle as positioned between the spherical stem and around the central stem; and (2) forward and horizontal motion, such as for being performed throughout various stages of the cleaning process. It is noted that the loading and unloading motion corresponds to the loading or unloading stage of the cleaning process of the system 100. Performance of the horizontal motion, forward motion, loading/unloading motion, or combination thereof may be executed simultaneously and/or sequentially as a cleaning motion.

By way of example, when the motion controller 218 executes the cleaning motion, the bottles loaded into the stabilizer 130 are caused to vary in position during a respective stage. As a result, the bottles receive variable water and/or vapor application during the cleaning process. Motion patterns performed may include vertical, horizontal, and/or diagonal motion of the bottles. In certain embodiments, the motion controller 218 may also perform rapid alternation between various directions of the cleaning motion, such as to generate shaking or vibration of the bottle. This motion results in the application of variable pressurized water or vapor to remove, loosen or dislodge stuck on or settled in residue from the bottles. Of note, the motion controller 218 may also be executed in conjunction with water pressure regulator 206 for further varying the pattern, intensity and flow of water as it contacts the bottle's surface.

In certain embodiments, the motion controller 218 is implemented as one or more dual or independently driven motion controllers. As such, the motion controller 218 may be programmatically configured to synchronize the action/motion of respectively placed threaded rollers (e.g., 440 and 450) for maintaining balance, control and execution of the stabilizer 130 throughout the various cleaning stages. In certain embodiments, additional extension shafts may be used instead of rollers to cause movement of the stabilizer 130. Still further, in certain embodiments, various tension devices, servo motors, amplifiers, stagers, actuators and the like may also be used for enabling motion. It is noted that various additional approaches for controlling the speed and direction of the stabilizer may be employed to accommodate different system 100 form factors.

With reference again to FIG. 2, a user interface controller 220 allows a user to control operation of the cleaning system 100. By way of example, the user interface controller 220 generates the interface in response to application programming interfaces (APIs) or other function calls corresponding to the user control panel 226. The user interface controller 220 is also configured to receive and interpret input signals as entered to a keypad, various buttons or other input means of the control panel 226. By way of example, the user interface controller 220 may receive a control signal from the keypad of the user control panel 226 and interpret the signal as activation of a “START” button. Based on this selection, the user interface controller 220 initiates execution of the programmatic instructions corresponding to the “START” signal.

Also, in one embodiment, a system controller 222 is configured to regulate the communication processes between the various other components of the cleaning system 100. For example, the system controller 222 may regulate timing between executions of respective stages of cleaning as performed in connection with a multi-stage controller. As another example, the system controller 222 may regulate the cycles of the water pump. The system controller may also be configured to trigger the execution of one or more motions via the motion controller 218, such as in response to a determined user input or condition being met.

In one embodiment, the multi-stage controller 224 regulates execution of various stages of the cleaning process. By way of example, the multi-stage controller 224 is configured to receive input provided by the user via a keypad or button of the user control panel 226 for affecting operation of the various stages. The inputs may include, for example, selection of a stage frequency, a number of repetitions or wash cycles, a length of time for executing a cleaning process, etc. The multi-stage controller 224 also operates in connection with several other components of the system—i.e., the cleaning agent application controller 214 or motion controller 218—for enabling proper execution of one or more cleaning stages.

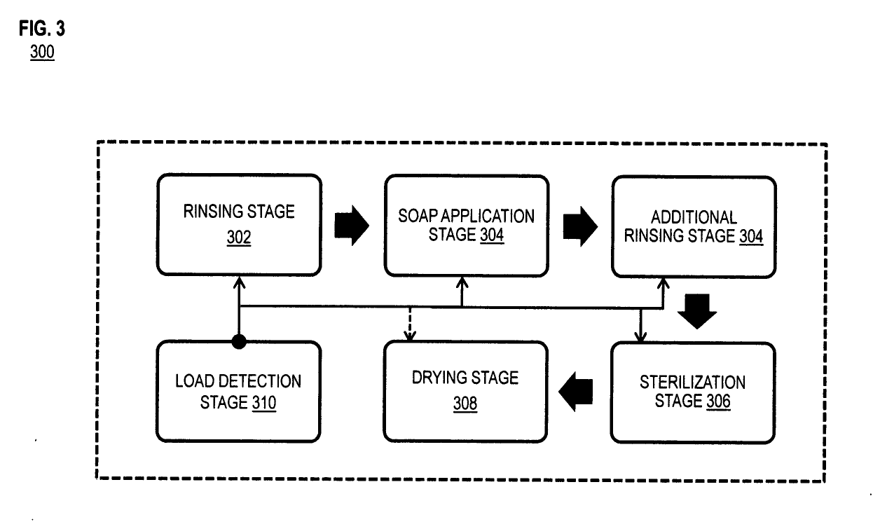

FIG. 3 is a flowchart of a process for enabling the cleaning of infant containers and other infant items, according to one embodiment. The process corresponds to that performed by the multi-stage controller of FIG. 2, with each step representing a particular cleaning stage. It is noted that the steps/stages 302-310 of the multi-stage cleaning process 300 may be performed in any suitable order, as well as combined or separated in any suitable manner. Also noted, execution of the various stages as performed with respect to bottles or other infant containers is also performed with respect to infant items placed in the accessories cage 500. Each stage may be executed concurrent with the performance of a cleaning motion (e.g., forward, horizontal, up/down motion) of the stabilizer 130 as facilitated by the motion controller 218. Feedback information pursuant to execution of a respective stage 302-310 may be presented to and interface of the user control panel.

In a step corresponding to a load detection stage 310, a determination is made that one or more bottles are ready to be cleaned. By way of example, the determination is based on the detected closing of a top enclosure (door 146) of the system 100 or on activation/closure of a contact based latch mechanism. In other instances, the determination may be based on a determined positional offset or variance of the stabilizer 130 from an at rest position, a detected load weight of the bottles to the stabilizer, etc. Of note, the load detection stage 310 may be associated with an alert of various types for indicating that the other stages of the cleaning process may proceed. This may include activation of a sound, light signal or message as rendered to the interface of the user control panel.

In another step corresponding to a rinsing stage 302, water is applied to the inner and outer surface of the loaded bottles. In certain embodiments, the rinsing stage 302 may be performed independently or concurrent with a soap application stage 304. During this stage 304, the detergent application controller applies soap, detergent or any other cleaning agent to the interior and exterior of the bottles. The cleaning agent may also be applied to items maintained in the accessories cage 500. It is noted that the cleaning agent application controller 314 may be configured to expel the soap through the stems of each sprayer or alternatively, by way of an independent sprayer or pressure based application means. In the latter scenario, a separate hosing system than that utilized for enabling the water ways 442 may extend from the soap reservoir 120 to enable soap application by the cleaning agent application controller 314.

Per a step corresponding to an additional rinsing stage 304, water is directed to the interior and exterior of the bottles again. The additional rinsing removes any soap as well as any loosened residue occurring as a result of concurrent execution of one or more cleaning motions. By way of example, in instances where the motion controller 218 executes a combination of rapid forward or horizontal (e.g., lateral) motions, or combinations thereof with respect to prior executed stages; particularly stuck on (grimy) residue is loosened or forced from the surface of the bottle. Thus, the additional rinsing stage facilitates removal of any loosened residue or enables another rinsing to occur to ensure cleanliness. Of note, the additional rinsing stage may be programmed for execution by the user to occur multiple times as part of the overall cleaning process executed by the multi-stage controller 224.

In a step corresponding to a sterilization stage 306, high temperature steam/vapor is directed towards the bottles and other infant items as a means of sanitization. In certain embodiments, the cleaning motion may or may not be applied during this stage. Per another step corresponding to a drying stage 308, the bottles and other infant items are allowed to dry. The drying stage 308 may include disabling the water pump 210, water temperature monitor 202 and/or water pressure regulator 204 to prevent the forcing of water or vapor onto the bottles or other items. The drying stage 308 may also include positioning the stabilizer 130 to its upmost position, thus minimizing the interaction or proximity between the stabilizer and the water application assembly 112. In the upmost position, the position of the stabilizer is equivalent to that required for initial loading of bottles and prior to detection of a load.

Still further, during the drying stage 308, one or more heating elements 230 may be activated to help evaporate any water droplets remaining on the bottles and other infant items. A drain valve or pressure release valve may also be activated to permit the extraction of excess water, residue and vapor build-up. In future embodiments, it is further contemplated that a fan mechanism or spinning mechanism may also be employed for facilitating the drying of bottles and other infant items.

Of note, the drying stage 308 may correspond to a depressurization stage of the multi-stage cleaning process, such as to enable the release of excess air or water pressure as accumulated during execution of the other stages of the cleaning process. In certain embodiments, the cleaning system 100 may be configured to prevent the opening of the door 146 (e.g., latch release) until the unit is depressurized to a specific threshold. As such, hot air and steam is released in a controlled manner as opposed to being released at once upon opening of the door 146. During this mode of operation, the control panel 226 may feature an indicator, i.e., a flashing warning, for alerting the user that the unit cannot be opened until after depressurization. Alternatively, in one embodiment, the cleaning system 100 may be configured to permit pressure release to occur throughout the cleaning process on an intermittent or as needed basis.

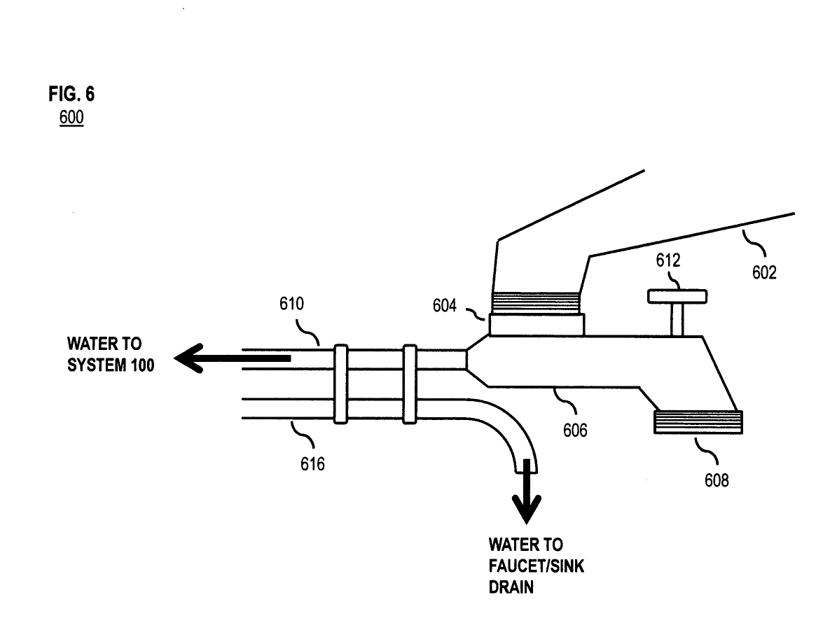

FIG. 6 is a diagram of a faucet configuration tool 600 for directing water to the cleaning system of FIG. 1, according to one embodiment. By way of example, the faucet configuration tool 600 may be employed to direct water from a water supply 602, through the water inlet, to the water way network and throughout the various central or spherical stems of the water application assembly 112. The configuration tool 600 can include a threaded connector 604 for mating with a complimentary threaded spout 602 or opening of a faucet or sink serving as the primary water supply. In certain embodiments, the configuration tool 600 includes a faucet extension element 606 for providing an additional spout 608 or opening in instances where the connector 604 is affixed to the primary faucet or sink.

Extending from one end of the extension element 606 is a water inlet hose 610. The water inlet hose 610 directs water received from the primary water supply 602 to the cleaning system 100. A water release valve 612 may also be configured to the extension element 606 for controlling the directional flow of water from the primary water supply 602. By way of example, a first position of the valve 612 may be set to enable the direction of water to the water inlet hose 610, while a second position of the valve 612 may be set to enable the direction of water to the additional spout or opening 608. It is noted that this approach enables a user to readily switch between supplying the cleaning system 100 with water and usage of the faucet 602. The user need not continually connect or disconnect the extension tool 606 to enable faucet use and can also use the faucet during operation of the cleaning system 100.

It is noted that the faucet configuration tool 600 as presented herein may be used optionally for providing water to the cleaning system 100. In another embodiment, the water inlet house 610 may be directly configured to the connector, such that the directional flow of water is restricted to only that of the cleaning system. In yet another embodiment, the cleaning system 100 may feature a water reservoir that can be filled up to a predetermined and detectable capacity. Under this scenario, water may be selectively allocated from the reservoir during the various cleaning stages and drained accordingly as required through a drain hose 616, such that the configuration tool is not needed.

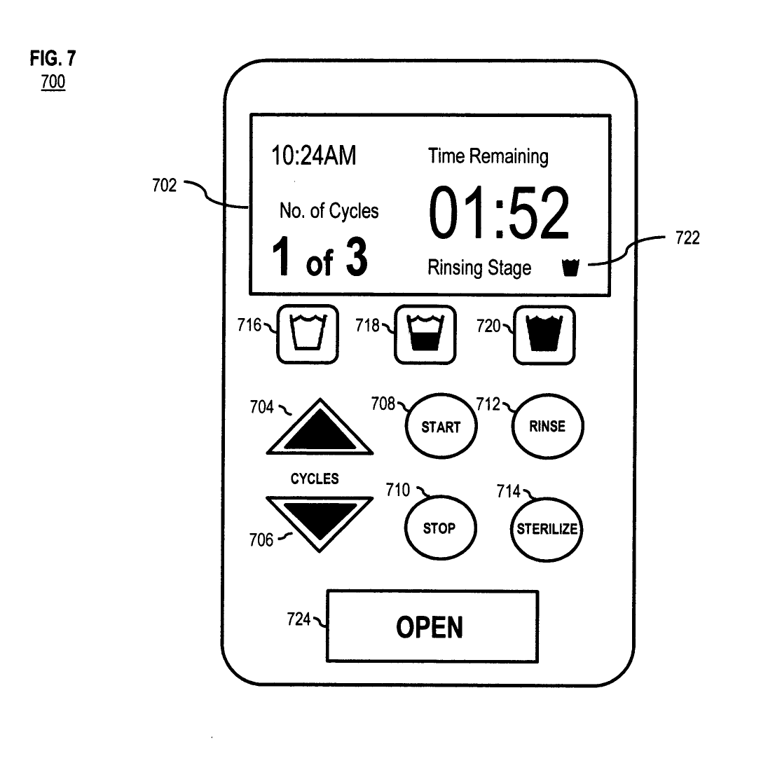

FIG. 7 is a diagram of a digital control panel for enabling user control of the cleaning system of FIG. 1, according to one embodiment. The user control panel 700 features a digital interface 702 for presenting status, functional or operational data to the user. The user control panel 700 also features various action buttons for enabling a user to provide input for affecting operation of the cleaning system 100. By way of example, the action buttons may include cycle time buttons 704 and 706 for affecting the number of repetitions of a particular stage of the multi-stage cleaning process (e.g., as shown in FIG. 3). Also, a “START” and “STOP” action button 708 and 710 respectively may be featured for enabling or disabling system 100 operations.

Still further, a “RINSE” action button 712 is featured for enabling a user to manipulate the number of additional rinsing stages performed by the cleaning system 100. A “STERILIZE” action button 714 enables the user to further manipulate the number of repetitions of the sterilization stage. It is noted, for example, that the user may further adapt and/or customize operation of the cleaning system 100 by selecting the “RINSE” or “STERILIZE” action buttons 712 and 714 respectively a number of times—i.e., up to a threshold of x times—to result in x number of corresponding additional rinsing and/or sterilization stages being performed. This may be useful to the user in instance where the infant container is heavily soiled.

In certain embodiments, the user control panel 700 may also feature various cleaning intensity action buttons 716-720. The cleaning intensity action buttons 716-720 allow the user to activate pre-determined multi-stage cleaning instructions based on their cleaning needs. Specifically, the cleaning intensity action buttons 716-720 enable a pre-determined number of additional stages, amount of time of execution, or combination thereof of the cleaning process to be carried out by the system 100. By way of example, a regular cleaning intensity action button 716 corresponds to programmatic instructions for carrying out a single cycle of each of the various stages of the multi-stage cleaning process. For this scenario, each stage is carried out in its entirety once, with each stage being performed based on a default time setting.

As another example, a medium cleaning intensity action button 718 corresponds to programmatic instructions for carrying out two cycles of one or more of the various stages of the multi-stage cleaning process (e.g., the additional rinsing and soap application stage). Under this scenario, each stage is carried out in its entirety twice, and the default time setting may be maintained. In another example, a heavy cleaning intensity action button 720 corresponds to programmatic instructions for carrying out three cycles of one or more of the various stages of the multi-stage cleaning process (e.g., the additional rinsing and sterilization stage). Under this scenario, each stage is carried out in its entirety three times, and the default time setting may be extended to an additional thirty seconds.

It is noted that the exemplary execution presented above may be adjusted by the user accordingly. For example, the user may establish the respective number of cycles, time limit and stages to be affected from selection of a given cleaning intensity action button. It is contemplated, in the one embodiment, the that user may configure the settings such as by pressing and holding a particular button 716-720 for a number of seconds, thus enabling the control panel 700 to receive input from the user accordingly. It is further contemplated that the user may be able to trigger execution of a cleaning motion of the device, such as when the user observes through the viewing pane of the system 100 that additional shaking or movement of the bottle is necessary to dislodge residue. This action may be activated by pressing one of the cleaning intensity action buttons 716-720 rapidly a number of times during operation of the cleaning system 100 or by way of a dedicated button (not shown) for inducing the cleaning motion (e.g., a “MOTION” or “SHAKE” action button).

The digital interface 702 of the user control panel 700 presents various status, functional and operational data regarding execution of the cleaning system. This includes, for example, a current time as provided by a digital clock, an amount of time remaining for execution of a particular stage of the multi-stage cleaning process, the name of the current stage of the multi-stage cleaning process being performed, an icon 722 representative of the selected level of cleaning intensity and data representative of the number of cycles performed or to be performed for the respective stage of the cleaning process. Of note, the digital interface 702 may present different data to accommodate different user needs, such as when the user is adjusting the settings of the cleaning intensity action buttons 716-720.

An “OPEN” button 724 may also be featured as part of or independent of the user control panel 700 for enabling the user to open the top enclosure or other established opening to the cleaning system 100. Activation of the “OPEN” button 724 may trigger the release of a latch or anchor mechanism established for maintaining the door in a locked/closed position. In certain embodiments, the “OPEN” button 724 may be implemented as an action button for generating control signals that affect the behavior of the latch. In other embodiments, the “OPEN” button 724 is itself implemented as a latch release mechanism (e.g., manual switch).

The exemplary techniques and system presented herein enable infant containers of various types, shapes and sizes to be cleaned through execution of a multi-stage cleaning process. One advantage offered by the techniques and system presented herein is that the multi-stage cleaning process may be customized by the user, i.e., by way of a user control panel that enables the manipulation of programmatic instructions for performing the cleaning. Another advantage is that the one or more stages of the multi-stage cleaning process may be carried out concurrent with the execution of a cleaning motion by the cleaning system. The cleaning motion enables variable movement of the one or more bottles and other infant items respective to a particular cleaning stage; thus enabling a more rigorous or effective cleaning action to occur without requiring the use of brushes, reaching tools and other instruments to remove built-up residue.

As another advantage, the cleaning system performs exemplary steps for: initiating an applying of water, one or more cleaning agents, or a combination thereof to one or more infant containers based, at least in part, on a selection of at least one cleaning stage; and causing, at least in part, motion of the one or more infant containers concurrent with the applying of the water, the one or more cleaning agents, or a combination thereof to the one or more infant containers. The motion corresponds to an upward movement, downward movement, lateral movement, shaking, or a combination thereof of the one or more infant containers during the cleaning stage.

The processes described herein for enabling the cleaning of infant containers and other infant items may be advantageously implemented via software, hardware, firmware or a combination of software and/or firmware and/or hardware. In addition, the various cleaning stages may be implemented via software, hardware, firmware or a combination of software and/or firmware and/or hardware. For example, various of the processes described herein, may be advantageously implemented via processor(s), Digital Signal Processing (DSP) chip, an Application Specific Integrated Circuit (ASIC), Field Programmable Gate Arrays (FPGAs), etc. Such exemplary hardware for performing the described functions is detailed below.

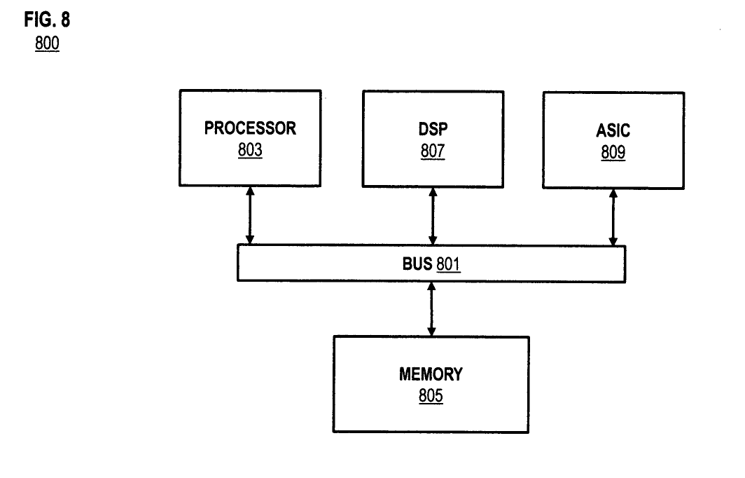

FIG. 8 illustrates a chip set or chip 800 upon which an embodiment of the invention may be implemented. Chip set 800 is programmed to enable the cleaning of infant containers and other infant items as described herein and includes, for instance, the processor and memory components described with respect to FIG. */ incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set 800 can be implemented in a single chip. It is further contemplated that in certain embodiments the chip set or chip 800 can be implemented as a single “system on a chip.” It is further contemplated that in certain embodiments a separate ASIC would not be used, for example, and that all relevant functions as disclosed herein would be performed by a processor or processors. Chip set or chip 800, or a portion thereof, constitutes a means for performing one or more steps of enabling the cleaning of infant containers and other infant items.

In one embodiment, the chip set or chip 800 includes a communication mechanism such as a bus 801 for passing information among the components of the chip set 800. A processor 803 has connectivity to the bus 801 to execute instructions and process information stored in, for example, a memory 805. The processor 803 may include one or more processing cores with each core configured to perform independently. A multi-core processor enables multiprocessing within a single physical package. Examples of a multi-core processor include two, four, eight, or greater numbers of processing cores. Alternatively or in addition, the processor 803 may include one or more microprocessors configured in tandem via the bus 801 to enable independent execution of instructions, pipelining, and multithreading. The processor 803 may also be accompanied with one or more specialized components to perform certain processing functions and tasks such as one or more digital signal processors (DSP) 807, or one or more application-specific integrated circuits (ASIC) 809. A DSP 807 typically is configured to process real-world signals (e.g., sound) in real time independently of the processor 803. Similarly, an ASIC 809 can be configured to performed specialized functions not easily performed by a more general purpose processor. Other specialized components to aid in performing the inventive functions described herein may include one or more field programmable gate arrays (FPGA) (not shown), one or more controllers (not shown), or one or more other special-purpose computer chips.

In one embodiment, the chip set or chip 800 includes merely one or more processors and some software and/or firmware supporting and/or relating to and/or for the one or more processors.

The processor 803 and accompanying components have connectivity to the memory 805 via the bus 801. The memory 805 includes both dynamic memory (e.g., RAM, magnetic disk, writable optical disk, etc.) and static memory (e.g., ROM, CD-ROM, etc.) for storing executable instructions that when executed perform the inventive steps described herein to enable the cleaning of infant containers and other infant items. The memory 805 also stores the data associated with or generated by the execution of the inventive steps.

While the invention has been described in connection with a number of embodiments and implementations, the invention is not so limited but covers various obvious modifications and equivalent arrangements, which fall within the purview of the appended claims. Although features of the invention are expressed in certain combinations among the claims, it is contemplated that these features can be arranged in any combination and order.

What is claimed is:

1. An infant container cleaning apparatus comprising:
a stabilizer having one or more arms for holding one or more infant containers;
a water application assembly for applying water, one or more cleaning agents, or a combination thereof to the one or more infant containers, the assembly having one or more stems that extend upward from a base of the water application assembly into one or more openings of the respective one or more infant containers, around the respective one or more infant containers, or a combination thereof, the one or more stems having positioned thereon a plurality of outlets for spraying water, the one or more cleaning agents, or a combination thereof to the inside and outside of the respective one or more infant containers; and
at least one motion controller for varying the position, movement, speed, or a combination thereof of the stabilizer during the applying of the water, the one or more cleaning agents, or a combination thereof to the respective one or more infant containers by the water application assembly.

2. An infant container cleaning apparatus of claim 1, further comprising:
a regulator for directing the water, the one or more cleaning agents, or a combination thereof to the water application assembly based, at least in part, on the at least one cleaning stage,
wherein the regulating includes (a) activating and/or deactivating a water supply, (b) activating and/or deactivating at least one dispenser of the one or more cleaning agents, (c) adapting a pressure setting associated with the water, the one or more cleaning agents, or a combination thereof, (d) adapting a temperature setting associated with the water, the one or more cleaning agents, or a combination thereof, or (e) a combination thereof.

3. An infant container cleaning apparatus of claim 1, further comprising:
an input controller for generating one or more control signals to initiate the water application assembly, the motion controller, the regulator, or a combination thereof based, at least in part, on one or more user inputs.

4. An infant container cleaning apparatus of claim 3, wherein the position, movement, speed, or a combination thereof of motion controller is based, at least in part, on the one or more user inputs.

5. An infant container cleaning apparatus of claim 4, wherein the one or more user inputs specify at least one cleaning stage, a number of cycles associated with the at least one cleaning stage, a time duration associated with the at least one cleaning stage, an intensity setting associated with the at least one cleaning stage, or a combination thereof.

6. A infant container cleaning apparatus of claim 5, wherein the at least one cleaning stage is associated with the applying of the water, the one or more cleaning agents, or a combination thereof to the one or more infant containers, a loading of the one or more infant containers, a drying of the one or more infant containers, or a combination thereof.

7. An infant container cleaning apparatus of claim 1, further comprising:
a housing for enclosing at least the stabilizer, the water application assembly, or a combination thereof during the applying of the water, the one or more cleaning agents, or a combination thereof to the one or more infant containers,
wherein the housing comprises a plurality of containment walls, at least one door adjacent to one or more of the plurality of containment walls, or a combination thereof.

8. An infant container cleaning apparatus of claim 7, wherein at least one of the plurality of containment walls has affixed thereto the water application assembly.

9. An infant container cleaning apparatus of claim 7, wherein at least one of the plurality of containment walls has positioned thereon at least one inlet for receiving water from a water supply, at least one outlet for evacuating water, residue, or a combination thereof to a drain, or a combination thereof.

10. An infant container cleaning apparatus of claim 1, further comprising:
a pump for pumping the water, the one or more cleaning agents, or a combination thereof through the water application assembly; and
one or more heating elements for increasing the temperature of the water, the one or more cleaning agents, or a combination thereof.

11. An infant container cleaning apparatus of claim 11, wherein the pump, the one or more heating elements, or a combination thereof are controlled by the regulator.

12. An infant container cleaning apparatus of claim 1, further comprising:
a compartment for maintaining one or more infant items, the compartment being enclosed by the housing,
wherein the water application assembly applies the water, the one or more cleaning agents, or a combination thereof to the one or more infant items maintained in the compartment during applying of the water, the one or more cleaning agents, or a combination thereof to the one or more infant containers.

13. An infant container cleaning apparatus of claim 1, wherein the motion controller further comprises:
one or more shafts, a first end of the one or more shafts being connected to the at least one motion controller, a second end of the one or more shafts being connected to one or more respective points along the stabilizer,
wherein the varying of the position, movement, speed, or a combination thereof of the stabilizer is based, at least in part, on a movement of the one or more shafts.

14. An infant container cleaning apparatus of claim 13, wherein the movement includes an upward movement, a downward movement, a lateral movement, a shaking, or a combination thereof.

15. An infant container cleaning apparatus of claim 1, wherein the one or more stems of the water application assembly include one or more central stems for applying the water, the one or more cleaning agents, or a combination thereof to the inside of the one or more infant containers, one or more spherical stems for applying the water, the one or more cleaning agents, or a combination thereof to the outside of the respective one or more infant containers, or a combination thereof.

16. An infant container cleaning apparatus of claim 15, wherein the one or more stems are connected to at least one channel in common, the at least one channel being connected to the regulator for receiving the water, the one or more cleaning agents, or a combination thereof.

17. A computer-readable storage medium carrying one or more sequences of one or more instructions which, when executed by one or more processors, cause an infant container cleaning apparatus to at least perform the following steps:
initiate an applying of water, one or more cleaning agents, or a combination thereof to one or more infant containers based, at least in part, on a selection of at least one cleaning stage; and
cause, at least in part, motion of the one or more infant containers concurrent with the applying of the water, the one or more cleaning agents, or a combination thereof to the one or more infant containers,
wherein the motion corresponds to an upward movement, downward movement, lateral movement, shaking, or a combination thereof of the one or more infant containers during the cleaning stage.

18. A computer-readable storage medium of claim 17, wherein the apparatus is caused further to:
receive one or more user inputs for indicating the at least one cleaning stage, a number of cycles associated with the at least one cleaning stage, a time duration associated with the at least one cleaning stage, an intensity setting associated with the at least one cleaning stage, or a combination thereof.

19. A computer-readable storage medium of claim 18, wherein the at least one cleaning stage is associated with the applying of the water, the one or more cleaning agents, or a combination thereof to the one or more infant containers, a loading of the one or more infant containers, a drying of the one or more infant containers, or a combination thereof.

20. A computer-readable storage medium of claim 17, wherein the motion causes variable application of the water, the one or more cleaning agents, or a combination thereof to one or more inner and outer surface points of the respective one or more infant containers.
